(19) United States
(12) Patent Application Publication
(10) Pub. No.: US 2016/0305923 A1
(43) Pub. Date: Oct. 20, 2016

(54) Consistometer and Methods of Measuring Cement Slurry Consistency

(71) Applicant: PT3 Design Inc., Kingwood, TX (US)

(72) Inventor: Chris A. Marvel, Porter, TX (US)

(73) Assignee: PT3 Design Inc., Kingwood, TX (US)

(21) Appl. No.: 14/690,402

(22) Filed: Apr. 18, 2015

(51) Int. Cl. G01N 33/38; G01N 11/14

(52) U.S. Cl. CPC G01N 33/383; G01N 11/14

(57) ABSTRACT

Consistometers for testing of cement slurry samples under pressure include a pressure vessel having a lower end opening. A piston is disposed within the lower end opening and is moveable to adjust the available volume of and pressure within a sample chamber within the pressure vessel.

### BACKGROUND OF THE INVENTION

1. Field of the Invention

The invention relates generally to the design of consistometers, such as those used to test wellbore cement.

2. Description of the Related Art

Construction of oil and geothermal wells typically requires the use of cements. Cement is most often used to secure wellbore casing within a wellbore. Cements are specifically designed for their properties and thickening times at elevated temperatures and pressures. For development and testing of well cements, pressure vessels are used to simulate downhole conditions. Pressurized consistometers are used to test consistency of cements. Cement consistency testing procedures have been standardized under the API's Specification for Materials and Testing for Well Cements (January 1982). To some extent, the equipment that is used to measure consistency has also been standardized under these rules. A cement sample can be tested by rotating a slurry cup containing a cement sample at a constant speed with a paddle extending into the slurry cup. The slurry cup is often rotated with magnetic drive. Resistance at the paddle to rotation of the sample is measured as the cement cures.

Traditionally, a “fill stick” is used to measure the level/amount of cement slurry within the slurry cup. The sample chamber is then pressurized by injection of liquid alongside the cement sample. The inventor has recognized that there is a possibility that the pressurizing liquid could mix with the cement sample being tested and contaminate the sample.

Conventional consistometer systems are limited in their ability to recreate downhole conditions to which cement slurry might be subjected during curing. For example, it might be desired to test a cement slurry sample at a pressure of 5000 psi and 300° F. In conventional systems, the test sample is pressurized first and thereafter is heated. The prior pressurization prevents the water in the sample from boiling. It typically takes much longer to heat up the sample than it does to pressurize the sample. Thus, the actual heat of the wellbore environment is not faithfully represented in the testing process.

### SUMMARY OF THE INVENTION

The invention provides improved designs for consistometers which incorporate mechanisms for dynamic adjustment of internal pressure within the pressure vessel. In addition, consistometers constructed in accordance with the present invention preclude potential contamination of the cement sample being tested by a pressurizing fluid. Consistometers in accordance with the present invention are also able to handle thermal expansion or contraction of test samples without exposing a pressure control system to damaging cement slurry. Such slurry can result in abrasive wear and plugged valves.

In a first described embodiment, the pressure vessel has a sample chamber, the available volume of which is governed by a piston that pressurizes the sample chamber. The position of the piston is governed by a screw drive. In certain embodiments, a controller controls the screw drive in response to pressure detected within the sample chamber.

In operation, the screw drive adjusts the available volume of the sample chamber as a cement slurry sample is placed into the sample chamber. The available volume of the sample chamber is adjusted to remove excess air and to achieve a desired pressure within the chamber. The sample chamber can be heated prior to or while placing the cement slurry sample into the sample chamber. Once the desired pressure within the sample chamber is achieved, a mixing paddle stirs the cement slurry sample while it sets and hardens. A torque sensor measures resistance of the paddle to rotation. Upon completion of the test, the piston can be removed from the housing of the consistometer, thereby improving the process of removing the cement slurry sample from the sample chamber.

According to a described alternative embodiment, a pressure vessel incorporates a piston that is biased by a spring and/or fluid pressure to provide an available volume within the sample chamber. The spring force and/or fluid pressure is varied to adjust the position of the piston and the available volume of the sample chamber.

### BRIEF DESCRIPTION OF THE DRAWINGS

For a thorough understanding of the present invention, reference is made to the following detailed description of the preferred embodiments, taken in conjunction with the accompanying drawings, wherein like reference numerals designate like or similar elements throughout the several figures of the drawings and wherein:

FIG. 1 is a side, cross-sectional view of a first exemplary consistometer constructed in accordance with the present invention.

FIG. 2 is a side, cross-sectional view of an alternative embodiment for a consistometer constructed in accordance with the present invention.

FIG. 3 is a flow diagram illustrating exemplary steps for testing cement slurry using consistometers in accordance with the present invention.

FIG. 4 is a side, cross-sectional view of an exemplary sample chamber with a cement slurry sample disposed therein.

FIG. 5 is a side, cross-sectional view of the sample chamber of FIG. 4, now with the available volume of the chamber having been adjusted by movement of the piston.

FIG. 6 is a side, cross-sectional view of the sample chamber of FIGS. 4-5 now with the upper opening having been closed.

### DETAILED DESCRIPTION OF THE PREFERRED EMBODIMENTS

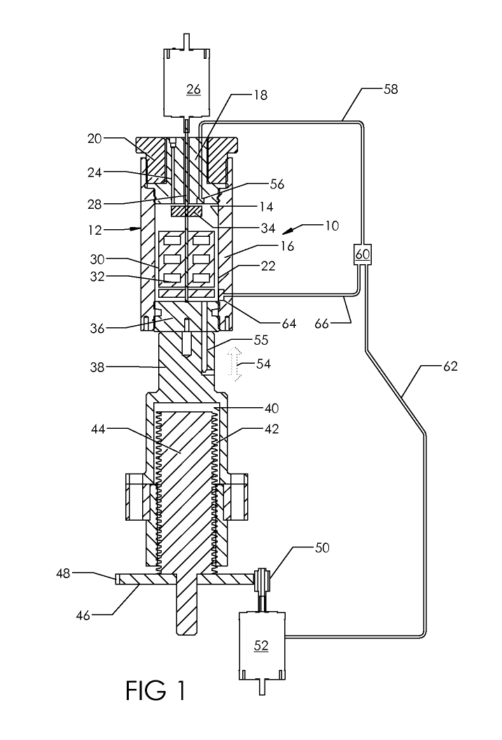

FIG. 1 depicts an exemplary consistometer 10 constructed in accordance with the present invention. The consistometer 10 includes a pressure vessel 12 which defines an interior sample chamber 14. In the depicted embodiment, the pressure vessel 12 includes a generally cylindrical housing 16. A plug member 18 encloses the upper end of the housing 12, being held in place by threaded cap 20. The pressure vessel 12 presents a lower end opening 22. A pressure port 24 is disposed through the plug member 18 and may be selectively opened and closed as needed to remove pressure from within the sample chamber 14. The pressure port 24 could also be used to introduce additional fluids into the sample chamber 14. Preferably, such additional fluids would not be fluids used to pressurize the sample chamber 14 but, rather, those intended as a part of the process for testing the sample. For example, certain tests are designed to simulate exposure of the cement sample to salt water and/or drilling fluids under mixing conditions. Alternatively, fluids might be introduced through the pressure port 24 to help remove the slurry sample from the sample chamber 14 once the testing has reached a finished state.

A motor 26 is coupled to shaft 28 which passes through the plug member 18. The motor 26 can be actuated to rotate shaft 28. A mixing paddle 30 is disposed within the sample chamber 14 and is affixed to shaft 28 so that rotation of the shaft 28 will also rotate the paddle 30. It is preferred that dynamic seals be provided between the shaft 28 and plug member 18 so that fluid sealing is maintained even as the shaft 28 is rotated. In certain embodiments, rectangular openings 32 are disposed through the paddle 30. A torque sensor 34 is mounted to the shaft 28. The torque sensor 34 may comprise a transducer that can detect resistance to rotation of the paddle 30 within the sample chamber 14.

A piston 36 is moveably disposed within the lower end opening 22 and seals against the opening 22. The piston 36 is affixed to arm 38. Arm 38 includes a blind bore 40 with a ring 42 contained therein. The ring 42 presents interior threading. A generally cylindrical threaded pin 44 is retained within the blind bore 40. The pin 44 has exterior threading that is complementary to the threading of the ring 42. The arm 38 and threaded pin 44 move telescopically with respect to each other as the pin 44 is rotates about its axis. A disc 46 is secured to the pin 44. Teeth are formed on the outer circumference 48 of the disc 44. The circumferential teeth are intermeshed with the teeth of toothed gear shaft 50. Gear shaft 50 is rotated by motor 52, thereby causing rotation of pin 44. Because the position of pin 44 and disc 48 is axially fixed, rotation of the disc 48/pin 44 will cause the arm 38 and affixed piston 36 to be moved axially in the directions illustrated by arrows 54 It is noted that the shaft 28 contacts the piston 36 during operation. A secondary fluid passage 55 may be formed within the piston 36 and arm 38 which will permit fluid communication with the sample chamber 14 from its lower end. Although not shown, it should be understood that the secondary fluid passage 55 is provided with suitable valves and controls to selectively close and open the secondary fluid passage 55 to fluid flow threrethrough. The secondary fluid passage 55 is useful for introduction of a given volume of fluid into the test system or to push out a given volume of original slurry. The secondary fluid passage 55 might also be useful in situations wherein foaming cements are used.

A pressure sensor 56 is incorporated into the plug member 18 and is operably associated (as indicated schematically at 58) with a processor or controller 60 which can receive a signal from the pressure sensor 56 that is indicative of the pressure within the sample chamber 14. The pressure sensor 56 may be a transducer. Alternatively, the pressure sensor 56 may be a fiber optic based sensor of a type known in the art or one of a number of other pressure sensing arrangements known in the art. The controller 60 is preferably a programmable digital processor with associated storage memory and programming which is capable of recording and/or displaying measured parameters for an operator. In certain embodiments, the controller 60 comprises a suitably programmed computer. In particular embodiments, the controller 60 is also operably associated with the motor 52, as indicated schematically by wiring 62, so that the motor 52 can be actuated by the controller 60 to adjust the position of the piston 36 within the housing 16. Also in particular embodiments, a position detector, shown schematically at 64, is retained within the pressure vessel 12 and is operable to detect the axial position of the piston 36 with respect to the pressure vessel 12 and provide a signal representative of this information to the controller 60 via transmission line 66.

The sample chamber 14 of the consistometer 10 may be heated using one (or more) of a number of techniques. A heated fluid (not shown) could surround the pressure vessel 12 to raise the temperature of the cement slurry sample within. Alternatively, heating filaments (not shown) could be disposed around or within the pressure vessel 12 and energized to heat the slurry sample. Because the consistometer 10 has a piston 36 that is adjustable to alter the available volume within the sample chamber 14, heating can occur prior to placing the slurry sample into the sample chamber 14. The available volume of the sample chamber 14 can also be adjusted to accommodate expansion of the slurry sample due to heating. As temperature increases and the available volume of the sample chamber 14 stays constant, the pressure within the sample chamber 14 will increase. The feature of having a piston 36 that is axially moveable within the lower end opening 22 of the pressure vessel 12 allows adjustment of the available volume of the sample chamber 14 to compensate for an increase in pressure resulting from temperature changes. Previous consistometers required fluid to be leaked or bled from the sample chamber in order to reduce pressure.

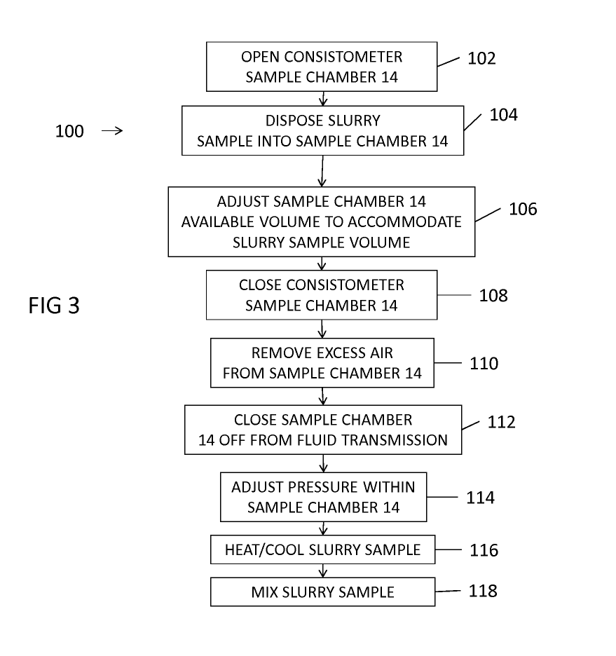

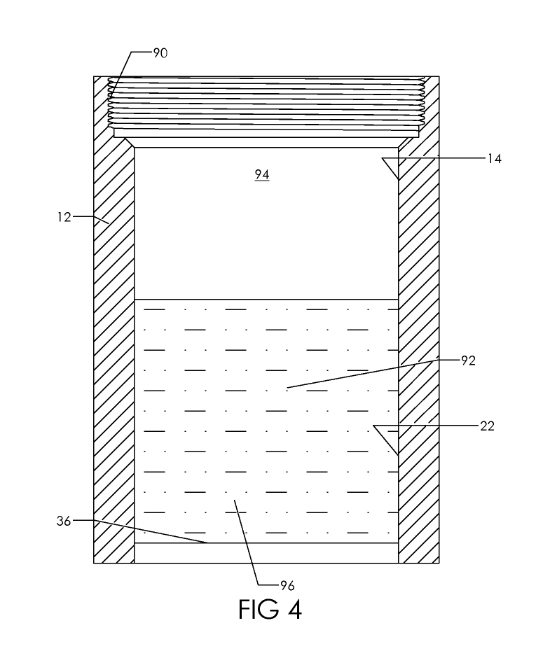

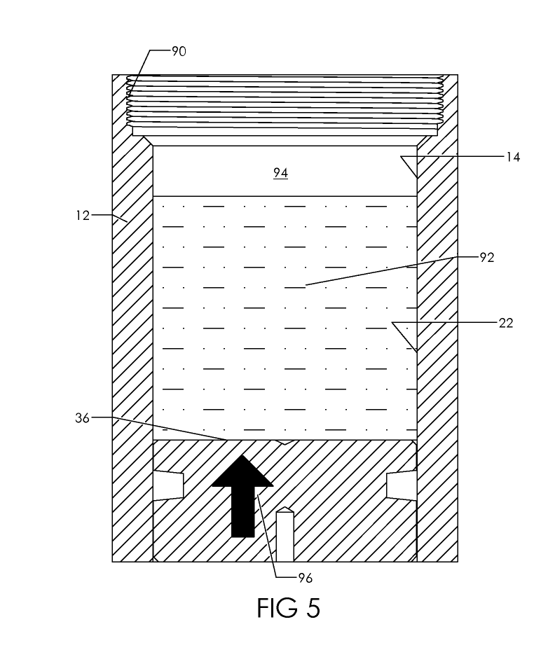

In operation, the consistometer 10 is used to test the consistency of a sample of a cement slurry. Further, the testing is performed to determine how quickly and how fully a particular cement slurry mix will set and cure under certain pressure and temperature conditions. According to an exemplary testing method, a cement slurry is first mixed together in a mix which it is desired to test. Typically, the amounts of dry ingredients for the slurry are measured, and the amount of required water is also measured. The water and dry ingredients are mixed together to create the slurry sample to be tested. With further reference to the process 100 illustrated in FIG. 3, the plug member 18 and the cap 20 are removed from the pressure vessel 12 to open the consistometer 10 (step 102), and the slurry sample is then poured into the sample chamber 14 (step 104). The position of the piston 36 is adjusted within the pressure vessel 12 so that the available volume of the sample chamber 14 is adjusted to accommodate the volume of slurry within the chamber 14 (step 106). According to currently preferred embodiments, the piston 36 is adjusted so that the volume of the slurry substantially fills the entire available space of the sample chamber 14. According to certain embodiments, the piston 36 is positioned by the controller 60 in response to position feedback from the position detector 64, which is capable of determining the axial position of the piston 36 with respect to the pressure vessel 12. FIGS. 4 and 5 illustrate the process of adjusting the available volume of the sample chamber 14. FIG. 4 illustrates a sample chamber 14 having an open upper end 90 because the plug member 18 and cap 20 have been removed. Slurry sample 92 has been poured into the sample chamber 14. There is a volume of excess air 94 within the chamber 14 above the slurry sample 92. FIG. 5 depicts the sample chamber 14 now that the piston 36 has been moved upwardly within the lower end opening 22, in the direction of arrow 96 so that the volume of excess air 94 is reduced.

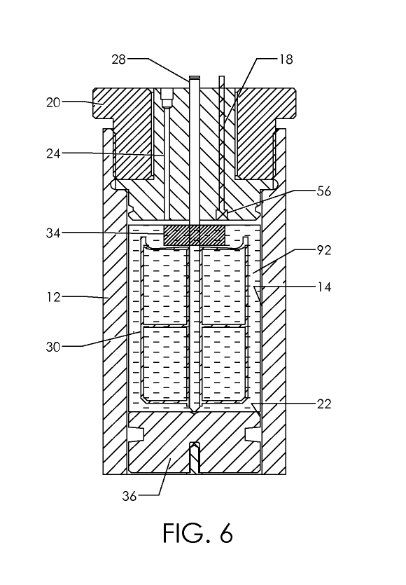

Next, the plug member 18 and cap 20 are secured to the pressure vessel 12 to close the sample chamber 14 of the consistometer 10 (step 108). When this is done, the mixing paddle 30 and torque sensor 34 are disposed into the slurry sample 92. FIG. 6 illustrates that fluid displacement of the slurry sample 92 by the inserted paddle 30, sensor 34 and shaft 28 helps to remove further excess air from the sample chamber 14. At this point, the pressure port 24 in the plug member 18 can be opened as the piston 36 is moved axially with respect to the pressure vessel 12 in order to remove any excess air from the sample chamber 14 (step 110).

The pressure port 24 can now be closed preventing fluid transmission to or from the sample chamber 14 (step 112). The piston 36 may now be adjusted, with respect to the housing 12, to increase or decrease pressure within the sample chamber 14, as desired (step 114). Pressure within the sample chamber 14 is detected by pressure sensor 56. The controller 60 then can adjust the position of the piston 36 so as to achieve a desired target pressure within the sample chamber 14. Typically, the target pressure will be a pressure that the cement slurry would be subjected to within a wellbore.

Heating or cooling may be applied to the sample chamber 14 to cause the slurry sample within to be subjected to a temperature or temperatures that would approximate the temperature or range of temperatures to which the cement slurry would be subjected in a wellbore (step 116). Because the consistometer 10 has the ability to adjust the available volume of the sample chamber 14, heating or cooling may be done prior to placement of the slurry sample in the chamber 14 or at any other time during the testing process.

The mixing paddle 30 is then rotated within the sample chamber 14 to mix the slurry sample as the slurry sample cures within the sample chamber 14 (step 118). The rate of paddle rotation is typically dictated by API Specification. During rotation of the mixing paddle 30, the torque sensor 34 measures resistance to paddle rotation within the sample chamber 14. Therefore, as the cement slurry sample begins to set and harden, the torque as measured by the torque sensor 34 will begin to increase. During the setting and hardening process, the temperature, pressure and torque (from torque sensor 34), or a user-defined combination of time, temperature, pressure and torque are measured and collated by the controller 60. The testing will be completed at a point determined by the user. For example, testing might be run until a certain torque reading is reached or, alternatively, run for a set amount of time regardless of the torque reading.

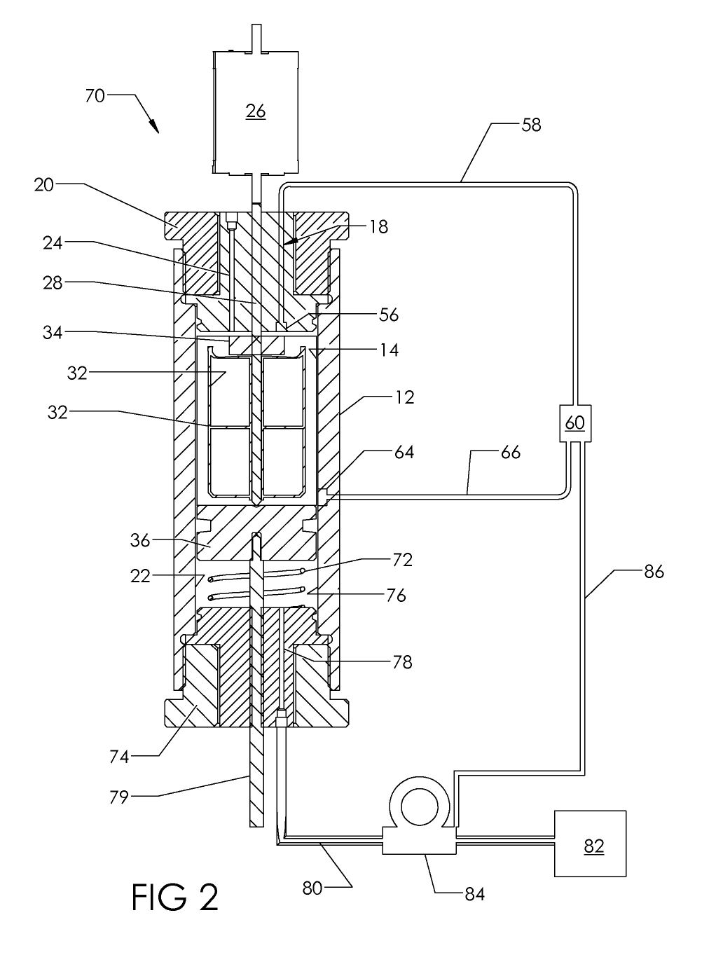

FIG. 2 illustrates an alternative embodiment for a consistometer 70 in accordance with the present invention. Except where otherwise described, the consistometer 70 is constructed and operates in the same manner as the consistometer 10 described previously. The piston 36 of the consistometer 70 is retained within the lower end opening 22 by a mechanical compression spring 72. In addition, lower end opening 22 of the pressure chamber 12 is enclosed by a lower cap assembly 74 so as to define a fluid spring chamber 76. The mechanical compression spring 72 is disposed within the fluid spring chamber 76 and contacts both the lower cap assembly 74 and the piston 36 so as to urge the piston 36 axially upwardly away from the lower cap assembly 74. In preferred embodiments, a fluid passage 78 is disposed through the lower cap assembly 74 and will selectively permit fluid communication to and from the fluid spring chamber 76. A shaft 79 extends downwardly from the piston 36 and through lower cap assembly 74. The function of the shaft 79 is to allow a position sensor (in place of sensor 64) to be attached outside of the pressure and temperature sensitive internal portions of the consistometer 70. A fluid conduit 80 interconnects a fluid supply 82 with the fluid passage 82. It is noted that, although the fluid supply 82 is indicated schematically as a specific box or component, the fluid supply 82 might merely be outside air. A fluid pump 84 is operably interconnected with the fluid conduit 80 to flow fluid into out of the fluid spring chamber 76 from the fluid supply 82, thereby displacing the piston 36 with respect to the pressure vessel. It is noted that the fluid spring chamber 76 is separated from the sample chamber 14 by the piston 36. Preferably, the fluid pump 84 is operably associated with the controller 60 via control line 86 so that operation of the fluid pump 84 may be controlled by the controller 60.

An exemplary method of operation for the consistometer 70, would largely follow the steps of process 100 described earlier. However, there would be difference in how individual steps are carried out. The position of the piston 36 is adjusted by flowing fluid into or out of the fluid spring chamber 76 rather than by operation of a screw drive.

Consistometers constructed in accordance with the present invention permit testing a cement slurry sample curing over a range of temperatures and pressures. The consistometers 10 and 70 allow handling of a cement slurry sample so that it can be tested over a range of temperatures and pressures which might mimic the conditions (and changes in conditions) which would be expected to occur during placement and curing of the cement being tested. Pressure within the sample chamber 14 can be adjusted during testing by adjusting the axial position of the piston 36 with respect to the pressure vessel 12. In addition, the temperature can be altered by increasing or decreasing the energy from heating media (i.e., fluids, heating coils) associated with the pressure vessel 12.

The invention allows for the removal of air entrapped within the pressure vessel 12 via pressure port 24. However, since there is no pressurizing fluid pumped into the sample chamber 14 in order to pressurize the slurry sample, there is no contaminated fluid to be dealt with in the event that pressure must be reduced within the sample chamber 14. In the event that it is necessary to reduce fluid pressure within the chamber 14, the piston 36 is merely moved axially downwardly with respect to the pressure vessel 12. Further, because the pressure vessel 12 has a lower end opening 22, a cement slurry sample can be more easily removed from the sample chamber 14 even if it were to inadvertently substantially set and harden within the sample chamber 14.

Those of skill in the art will recognize that numerous modifications and changes may be made to the exemplary designs and embodiments described herein and that the invention is limited only by the claims that follow and any equivalents thereof.

What is claimed is:

1. A consistometer for testing consistency of a cement slurry sample, the consistometer comprising:
a pressure vessel defining a sample chamber within having an available volume;
the pressure vessel having a lower end opening;
a piston disposed within the lower end opening and axially moveable with respect to the housing to alter the available volume of the sample chamber; and
a mixing paddle that is disposed within the sample chamber and rotated to stir the cement slurry sample.

2. The consistometer of claim 1 wherein the piston is axially moveable by a screw drive.

3. The consistometer of claim 1 wherein the piston is axially moveable by fluid pressure.

4. The consistometer of claim 4 wherein the piston is axially moved by fluid pressure by further flowing a fluid from a fluid supply into or out of a fluid spring chamber which is separate from the sample chamber, fluid from the fluid supply displacing the piston with respect to the pressure vessel.

5. The consistometer of claim 1 further comprising a programmable controller which governs axial movement of the piston with respect to the pressure vessel.

6. The consistometer of claim 5 further comprising a pressure sensor to detect pressure within the sample chamber and provide a signal representative of the detected pressure to the controller.

7. The consistometer of claim 5 further comprising a position detector to detect the axial position of the piston with respect to the pressure vessel and provide a signal representative of the axial position to the controller.

8. The consistometer of claim 5 wherein the controller adjusts the axial position of the piston in response to at least one of a) detected pressure within the sample chamber and b) detected position of the piston with respect to the pressure vessel

9. A consistometer for testing consistency of a cement slurry sample, the consistometer comprising:
a pressure vessel defining a sample chamber within having an available volume;
the pressure vessel having a lower end opening;
a piston disposed within the lower end opening and axially moveable with respect to the housing to alter the available volume of the sample chamber;
a mixing paddle that is disposed within the sample chamber and rotated to stir the cement slurry sample; and
a programmable controller which governs axial position of the piston with respect to the pressure vessel.

10. The consistometer of claim 9 wherein the piston is axially moveable by a screw drive.

11. The consistometer of claim 9 wherein the piston is axially moveable by fluid pressure.

12. The consistometer of claim 11 wherein the piston is axially moved by fluid pressure by further flowing a fluid from a fluid supply into or out of a fluid spring chamber which is separate from the sample chamber, fluid from the fluid supply displacing the piston with respect to the pressure vessel.

13. The consistometer of claim 9 wherein the controller adjusts the axial position of the piston in response to at least one of a) detected pressure within the sample chamber and b) detected position of the piston with respect to the pressure vessel.

14. The consistometer of claim 9 further comprising a pressure sensor to detect pressure within the sample chamber and provide a signal representative of the detected pressure to the controller.

15. The consistometer of claim 9 further comprising a position detector to detect the axial position of the piston with respect to the pressure vessel and provide a signal representative of the axial position to the controller.

16. A method of testing a sample of cement slurry for consistency, the method comprising the steps of:
disposing the slurry sample into a sample chamber, the sample chamber being defined within a pressure vessel having a lower open end, a moveable piston being disposed within the lower open end so as to define an available volume for the sample chamber;
closing an upper opening of the pressure vessel to close off the sample chamber;
adjusting pressure within the sample chamber by axially moving the piston with respect to the pressure vessel; and
mixing the slurry sample within the sample chamber.

17. The method of claim 16 further comprising the step of adjusting the available volume of the sample chamber to accommodate the slurry sample prior to closing the upper opening of the pressure vessel.
